(19) United States
(12) Patent Application Publication
(10) Pub. No.: US 2015/0011778 A1
(43) Pub. Date: Jan. 8, 2015

(54) USE OF TRIFLUOROACETAMIDE FOR N-TERMINAL PROTECTION

(71) Applicant: The Regents of the University of California, Oakland, CA (US)

(72) Inventors: Scott Lokey, Santa Cruz, CA (US); Rushia Turner, Santa Cruz, CA (US)

(21) Appl. No.: 14/379,718

(22) PCT Filed: Feb. 19, 2013
(86) PCT No.: PCT/US13/26760

§ 371 Date: Aug. 19, 2014

Related U.S. Application Data
(60) Provisional application No. 61/600,655, filed on Feb. 19, 2012.

(51) Int. Cl. C07K 1/06; C07K 1/04

(52) U.S. Cl. CPC C07K 1/064; C07K 1/04
USPC 548/335.5; 562/567; 562/560; 562/568; 562/561; 562/450; 548/535; 548/497; 548/496

(57) ABSTRACT

This disclosure teaches the novel use of trifluoroacetamide for N-terminal protection. The disclosure also teaches novel compositions and chemical structures associated therewith. These methods and compositions are useful for site-specific methylation of peptide backbone amides, performed, for example, to modulate the pharmacokinetic properties of peptide drugs.

### FIELD OF THE INVENTION

Protecting groups during solid phase peptide synthesis chemistry

### BACKGROUND

Peptides are synthesized by coupling the carboxyl group or C-terminus of one amino acid to the amino group or N-terminus of another. Due to the possibility of unintended reactions, protecting groups are usually necessary. Fmoc (Fluorenylmethyloxycarbonyl chloride) is currently used as a protecting group for solid phase peptide chemistry. This is a billion dollar market. This disclosure teaches a new and highly advantageous protecting group for solid phase peptide chemistry.

### BRIEF DESCRIPTION OF THE INVENTION

The amine protecting group is central to solid phase peptide synthesis. The chemistry for its removal must be efficient, fast, and orthogonal to both the linkage to the solid phase as well as the various side chain protecting groups. Permanent side chain protecting groups are typically benzyl or benzyl-based groups. Final removal of the peptide from the linkage occurs simultaneously with side-chain deprotection with anhydrous hydrogen fluoride (HF) via hydrolytic cleavage.

The common amine protecting groups in peptide synthesis are the acid-labile Boc (tert-butyloxycarbonyl), and the base-labile Fmoc (Fluorenylmethyloxycarbonyl chloride).

The Boc group has enjoyed widespread use as an efficient protecting group, although it suffers the limitation that cleavage from the solid phase must be performed with the highly dangerous reagent hydrogen fluoride (HF), requiring special training and a closed, glass-free apparatus. Therefore the Fmoc group emerged as the alternative to Boc.

Although Fmoc has become established as the primary chemistry used in peptide synthesis, the Fmoc group is large (mw=223) and relatively expensive, and few of the Fmoc amino acids are crystalline. The poor atom economy of the Fmoc group makes the synthesis of commercial peptides with Fmoc intrinsically inefficient.

The inventors have invented a new method for amine protection during solid phase peptide synthesis (SPPS) using the trifluoroacetamide (Tfac) group. While the Tfac group has been used as an amine protecting group for decades, and has even been used as an amine protecting group in solution phase peptide synthesis, its use in solid phase peptide synthesis is, we believe, novel.

The novelty of the method of the invention lies in the previously undisclosed and unrecognized use of the trifluoroacetamide (Tfac, also called Tfa) as an alternative to Fmoc in SPPS. This is non-trivial because there is a body of literature on protection group chemistry claiming that the Tfac group is hyper labile, yet the Tfac group is stable to virtually all of the reaction conditions introduced in SPPS.

Also novel is the removal of the Tfac group from a resin-bound state using sodium borohydride in a mixed ethanol/tetrahydrofuran solvent system.

Also novel is the installation of the Tfa group to a resin-bound peptide that employs a modified version of the solution phase procedure (as indicated below).

Lastly, also novel is the use of the Tfac group to facilitate site specific N-methylation of resin-bound peptides under Mitsunobu conditions, particularly in the context of amino acids with nucleophilic side chains like lysine, tryptophan, and arginine.

The Tfac group has been used for N-terminal protection in solution phase peptide synthesis, and it has been used to protect the side chain of Fmoc-Lysine, but it has not been used in SPPS, it has not been removed from resin-bound state using sodium borohydride in a mixed ethanol/tetrahydrofuran solvent system, and it has not been used to facilitate site specific N-methylation of resin-bound peptides under Mitsunobu conditions. The present invention has significant distinctions and advantages over the art.

The current invention employs the Tfac group as an atom-economical protecting group for solid phase peptide chemistry that can easily be removed by NaBH4 in THF/EtOH, conditions that are orthogonal to side chain protecting groups as well as the common resin linkage based on trityl esters.

This disclosure teaches the novel use of trifluoroacetamide (Tfac) for N-terminal protection. The disclosure also teaches novel compositions and chemical structures associated therewith.

### BRIEF DESCRIPTION OF THE FIGURES

The figures are integrated into the specification and include:

FIG. 1: Scheme 1a: Solution phase synthesis of amino acid trifluoroacetamides

FIG. 2: Scheme 2: Selective N-terminal methylation of peptides containing an N-terminal trifluoroacetamide.

FIG. 3: Scheme 3: A method for removal of a Tfac group from a protected compound.

FIG. 4: Trifluoroacetamide structure and physical characteristics

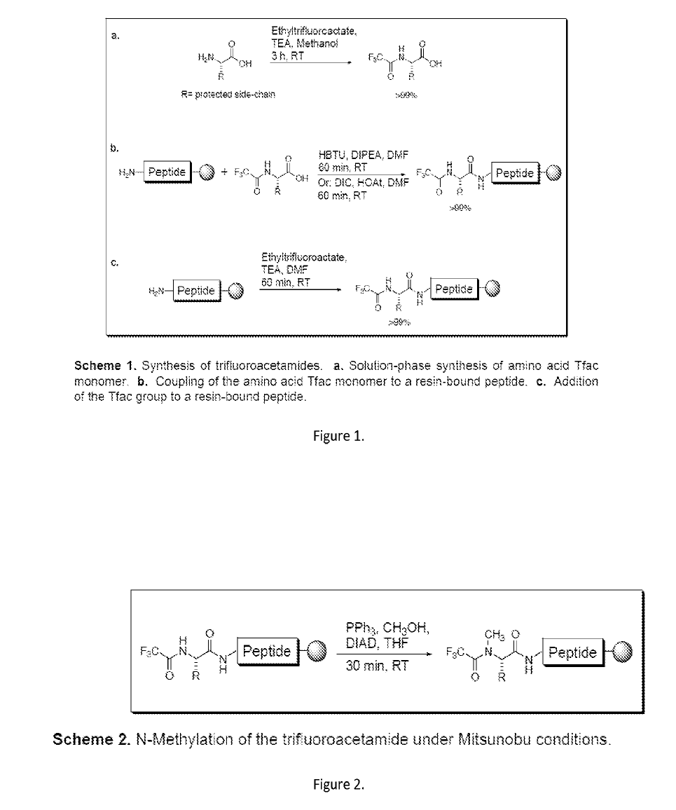

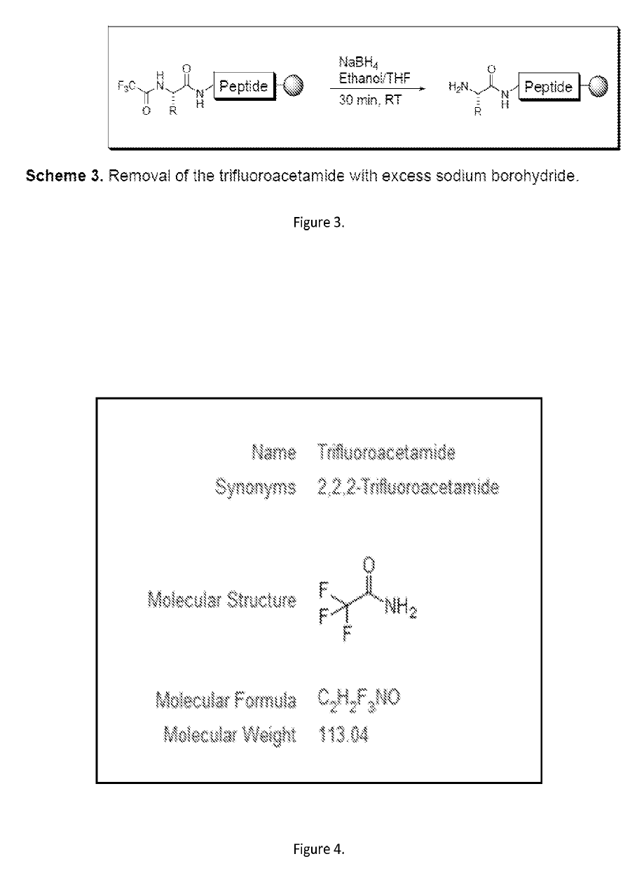

### GENERAL REPRESENTATIONS CONCERNING THE DISCLOSURE

All scientific papers, publications, patent documents and other disclosures mentioned herein are hereby incorporated by reference for all purposes. Particularly incorporated is Weygand & Frauendorfer Chem. Ber. (1970) 103, 2437.

The embodiments disclosed in this specification are exemplary and do not limit the invention. Other embodiments can be utilized and changes can be made. As used in this specification, the singular forms “a”, “an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). Where reference is made herein to “first” and “second” features, this is generally done for identification purposes; unless the context requires otherwise, the first and second features can be the same or different, and reference to a first feature does not mean that a second feature is necessarily present (though it may be present). Where reference is made herein to “a” or “an” feature, this includes the possibility that there are two or more such features. This specification incorporates by reference all documents referred to herein and all documents filed concurrently with this specification or filed previously in connection with this application, including but not limited to such documents which are open to public inspection with this specification.

Tfac=trifluoroacetamide (also abbreviated to “Tfa” in some of the literature)

Boc=tert-butyloxycarbonyl

Fmoc=Fluorenylmethyloxycarbonyl chloride

SPPS=solid phase peptide synthesis

The term “derivative” or “derivative compound” refers to a compound having a chemical structure that contains a common core chemical structure as a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

The term “fragment” refers to a part of a larger whole, for example a fragment of a molecule may be any dissociated part of that molecule, regardless of size.

The term “specie” or “group” when used to describe an “R” group in a chemical formula, is used to mean any chemical compound, sub-compound or substituent that may chemically interact with (covalently, ionically or by Van der Waal's forces) another molecule or group such as shown on a chemical formula.

When a “terminus” or “terminal group” is discussed as having a substituent, side-chain, group or moiety attached, that substituent, side-chain, group or moiety may equally be present at one or more termini or at side locations along the length of the molecule.

Where substitutions are mentioned, sometimes in connection with variable “R” groups as shown in the figures, the substituent groups may be selected from, for example, the following: hydrogen, hydroxyl, carboxylate, alkane, alkene or alkyne groups, substituted or unsubstituted heteroatom, alkyl, alkenyl, alkanoyl, aryl, aroyl, aralkyl, alkylamino cycloalkyl, heterocycloalkyl, heteroaryl, or halogen, azido, fluorophore or polypeptide. In certain embodiments the substituent group may comprise branched or un-branched C1-C18 alkyl, C1-C18 substituted alkyl, C1-C18 alkenyl, C1-C18 acyl, amino, substituted amino, wherein the alkyl, alkenyl or acyl is linear or branched, and optionally substituted with a hydroxyl, an ester and its derivatives, 5 a carboxyl and its derivatives. In a particular embodiment, Any R group may be a lower hydrocarbon substituted with alkoxy, substituted alkoxy, imidate, aiylthio, or (substituted aryl)thio. In other embodiments, Any R group may be a lower alkyl selected from methyl, ethyl, propyl, isopropyl, butyl, isobutyl, terabutyl and pentyl. In other embodiments, Any R group may be a lower alkenyl selected from vinyl, substituted vinyl, ethynyl, or substituted ethynyl. In other embodiments, Any R group may be a lower alkanoyl selected from formyl, acetyl. propionyl, isopropionyl, butyryl, isobutyryl, tert-butyryl, valeryl, pivaloyl, caproyl, capryl, lauryl, myristyl, palmityl, stearyl, arachidyl, stilligyl, palmitoyl, oleyl, linolenyl, and arachidonyl. In other embodiments, Any R group may be lower aryl selected from phenyl, p-tolyl, pchlorophenyl, p-aminophenyl, p-nitrophenyl, p-anisyl. In yet other embodiments, Any R group may be a lower aroyl selected from benzoyl and naphthoyl. In other embodiments, Any R group may be a lower aralkyl selected from benzyl, benzhydryl, p-chlorobenzyl, m-chlorobenzyl, p-nitrobenzyl, benzyloxybenzyl, or pentaflourobenzyl. In certain other embodiments, Any R group may be a lower alkylamino is selected from monoalkylamino, monoaralkylamino, dialkylamino, diaralkylamino, and benzylamino.

### DETAILED DESCRIPTION OF THE INVENTION

This disclosure teaches the novel use of trifluoroacetamide (Tfac) for N-terminal protection. Embodiments include:

The novel use of trifluoroacetamide (Tfac) for N-terminal protection of amino acids, dipeptides, peptides, and ploypeptides; the novel structures disclosed herein and their variants and substituted derivatives; compositions comprising the structures disclosed herein and their variants and substituted derivatives.

A novel method for solid phase peptide synthesis (SPPS) wherein N-terminal protection of amino acids is achieved using trifluoroacetamide (Tfac), and wherein deportection is achieved using sodium borohydride (NaBH4) in THF/EtOH.

A method comprising using trifluoroacetamide (Tfac) for N-terminal protection of amino acids, dipeptides, polypeptides and their variants and derivatives, for example site-specific methylation of peptide backbone amides to modulate the pharmacokinetic properties of peptide drugs.

A method comprising the removal of the Tfac group from a resin-bound state using sodium borohydride in a mixed ethanol/tetrahydrofuran solvent system.

A method comprising the use of the Tfac group to facilitate site specific N-methylation of resin-bound peptides under Mitsunobu conditions, particularly in the context of amino acids with nucleophilic side chains like lysine, tryptophan, and arginine.

Solution phase synthesis of amino acid trifluoroacetamides for subsequent use in SPPS by the steps of stirring the amino acid in methanol and triethylamine followed by the slow addition of ethyl trifluoroacetate (Scheme la), wherein the reaction is complete in no more than 3 hours and yields >99% of pure product after liquid-liquid extraction.

A method for selective N-terminal methylation of peptides containing an N-terminal trifluoroacetamide by the steps of treatment with an excess of methanol and triphenylphosphine in tetrahydrofuran, followed by the addition of excess di-isopropyl azadicarboxylate (Scheme 2) to yield >99% N-methylation within no more than 30 min

A method for removal of a Tfac group from a protected compound (e.g., and amino acid) within no more than 60 min by treatment with excess sodium borohydride (NaBH4) in a mixed solvent system of THF (tetrahydrofuran aka 1,4-epoxy-butane) and ethanol (1:1 v/v) (Scheme 3).

Another specific embodiment encompasses a method for the selective N-terminal methylation of a peptide containing an N-terminal trifluoroacetamide, the method comprising treatment of the peptide with an excess of methanol and triphenylphosphine in tetrahydrofuran, followed by the addition of excess diisopropyl azadicarboxylate so as to yield >99% N-methylation product within 30 minutes (as shown in Scheme 2). Step-by-step, the method is as follows: Procedure for Mitsunobu methylation of resin-bound, Tfa-protected peptides: Reactions were conducted in polypropylene synthesis vials equipped with a fritted disk and a teflon stopcock. Methanol and DIAD were dried via storage over flame-dried, four-angstrom molecular sieves for 24 h prior to use. Before the start of each reaction, the resin (˜100 mg of resin, 0.16 mmol of peptide) was rinsed with three small volumes of anhydrous THF in order to remove trace amounts of water. Triphenylphosphine (0.198 g, 5 eq) was transferred to a small glass vial and dissolved in ˜1 mL of anhydrous THF. Anhydrous methanol was added (65 μL, 10 eq) and the resulting solution was transferred to the synthesis vial containing the resin-bound Tfa-protected peptide. DIAD was added dropwise (5 eq, 158 μL) with vigorous agitation to the resin to facilitate both thorough mixing and dissipation of heat. After the DIAD addition was complete, the synthesis vial was capped and placed on a shaker to facilitate agitation during the 30 min reaction time. With amino acids wherein the side chain contained a bulky protection group, the process was repeated once or twice in order to push the reaction to completion. Even in cases like N-terminal methylation of lysine, arginine, and tryptophan repetition of the procedure did not introduce side-chain methylation or inadvertent Tfa-deprotection.

A further specific embodiment encompasses a method for solid phase peptide synthesis (SPPS) comprising N-terminal protection of an amino acid by the binding of a trifluoroacetamide (Tfa) to the N-terminus of the amino acid, and wherein deprotection of the amino acid is achieved by the removal of the Tfac group, within no more than 60 minutes by treatment with excess sodium borohydride in a mixed solvent system of THF and ethanol (1:1 v/v) (Scheme 3).

Trifluoroacetylation of a resin-bound peptide: On-resin N-terminal trifluoroacetylation was carried out by treating the deprotected N-terminal amine with DBU (12 eq) and ETFA (10 eq) in minimal DMF. The reaction was agitated for 60 min After each reaction, the resin was washed thoroughly with DMF (3×5 mL), DCM (3×5 mL) and again with DMF (3×5 mL). The reaction time can be reduced to ten minutes through the use of a microwave reactor.

Procedure for coupling a Tfa-protected amino acid to a resin-bound peptide: The N-terminal amine of a resin bound peptide was deprotected and the resin was washed with DCM (0.1 g of resin; 0.16 mmol). The Tfa-protected amino acid was dissolved in THF to a concentration of 100 mg/mL (3 eq, 0.109 g). Diisopropyl carbodiimide (DIC) was added to this solution (3 eq, 74 μL) followed by hydroxy azabenzotriazole (HOAt, 3 eq, 65 mg). The solution was sonicated for 60 s then added to the resin. The resin was agitated for 60 min then drained and rinsed with DCM and DMF.

Procedure for Tfa deprotection: For each deprotection, the resin-bound, Tfa-protected peptide was transferred to a polypropylene synthesis vial (˜0.1 g of resin, 0.16 mmol) and rinsed with three small volumes of anhydrous THF in order to remove trace amounts of water. The Tfa group was removed by suspending the resin (˜0.1 g, 0.16 mmol) in a mixture of anhydrous ethanol and anhydrous THF (˜1 mL, 1:1 v/v). Sodium borohydride was added (10 eq, 6 mg), the vial was capped, and the mixture was agitated vigorously. The reaction generated significant hydrogen gas and the reaction vial became pressurized as a result. It was necessary to loosen the cap and allow the reaction to sit undisturbed for 30 min After 30 min the solution was drained from the synthesis vial and the resin was rinsed with methanol and DCM. This procedure afforded 100% removal of the Tfa group within 30 min

The disclosure also teaches novel compositions and chemical structures associated therewith.

Name Trifluoroacetamide

Synonyms 2,2,2-Trifluoroacetamide

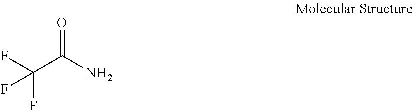

Molecular Formula C₂H₂F₃NO

Molecular Weight 113.04

These methods and compositions are useful for site-specific methylation of peptide backbone amides, performed, for example, to modulate the pharmacokinetic properties of peptide drugs.

This disclosure teaches a new method of using a protecting group for solid phase peptide chemistry that provides an alternative to the currently used Fmoc (Fluorenylmethyloxycarbonyl chloride), which is a billion dollar market. The protecting group is trifluoracetamide (Tfac) and the use of this compound for N-terminal protection is novel.

Also novel are compositions comprising amino acids bound to trifluoracetamide and methods comprising protection of amino acids, dipeptides, polypeptides and their variants and derivatives, for example site-specific methylation of peptide backbone amides to modulate the pharmacokinetic properties of peptide drugs.

Trifluoroacetamide (Tfac) provides a hyperlabile protecting group with limited orthogonality and has several important advantages over other N-terminal protection groups, discussed below.

The inventors investigated Tfac as an alternative to other N-terminal protection groups like the o-nitrobenzenesulfonamide (o-NBS) that facilitate the site-specific methylation of backbone amides. In comparison to some of the commonly used amine protecting groups used in SPPS, Tfac proves inexpensive and easy to install, and it improves the atom economy of solid phase peptide synthesis (SPPS). Very importantly, the electron deficient Tfac enhances the acidity of the amide proton thereby enabling efficient site-specific methylation of the Tfac nitrogen under standard Mitsunobu reaction conditions.

The synthesis of N-terminal Tfac amino acids can be done in both the solution phase and on solid phase (Scheme 1).

The solution phase synthesis of amino acid trifluoroacetamides for subsequent use in SPPS is accomplished by stiffing the amino acid in methanol and triethylamine followed by the slow addition of ethyl trifluoroacetate (Scheme la). The reaction is complete in 3 hours and yields >99% of pure product after a simple work-up consisting of liquid-liquid extraction.

The Tfac amino acid monomer is coupled to a resin-bound peptide using standard uronium or carbodiimide coupling agents (Scheme 1b). The trifluoroacetylation of resin-bound peptides can also be accomplished by protection group exchange. Upon cleavage of the N-terminal protecting group, the Tfac group is introduced by treating the resin with a large excess of triethylamine in DMF followed by the slow addition of excess ethyltrifluoroacetate (Scheme 1c). The reaction is complete in 60 min and affords >99% yields of pure trifluoroacetamide product.

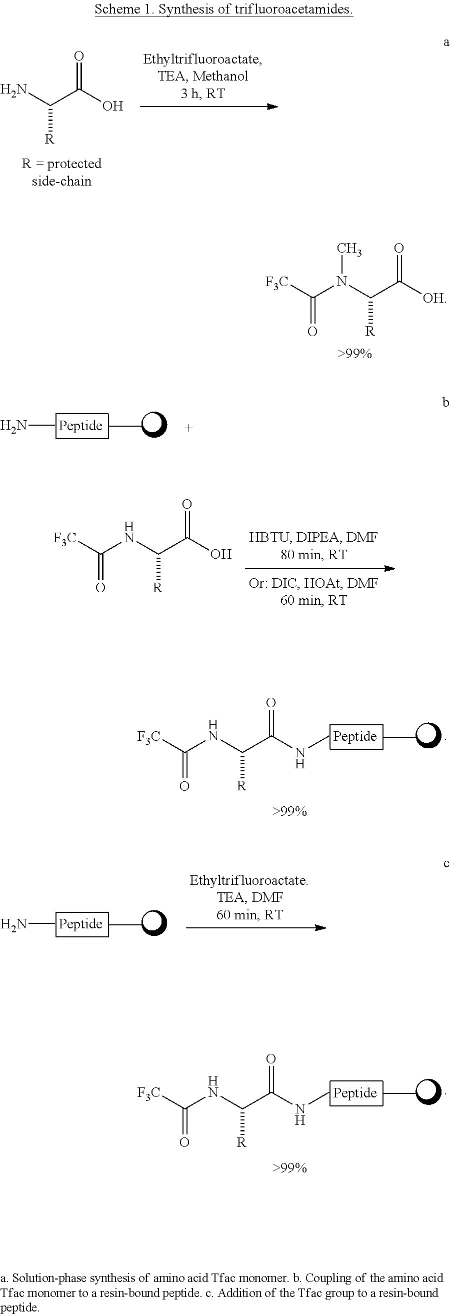

The development of mild and reliable methods for site-specific methylation of peptide backbone amides is highly desirable to researchers seeking to modulate the pharmacokinetic properties of peptide drugs. The methods previously reported in the literature have drawbacks that include incomplete methylation, promiscuous methylations, or the use of reagents that are problematic or otherwise objectionable.

Trifluoroacetamide is a suitable substrate for methylation under Mitsunobu conditions because the electron-deficient trifluoroacyl group enhances the acidity of the amide proton and stabilizes the trifluoroamidate anion.

The inventors found that peptides containing an N-terminal trifluoroacetamide could be selectively methylated at the N-terminus by treatment with an excess of methanol and triphenylphosphine in tetrahydrofuran, followed by the addition of excess diisopropyl azadicarboxylate (Scheme 2). These conditions yield >99% N-methylation within 30 min, and are shown to eliminate the problems associated with nonspecific methylations that are observed in other methods.

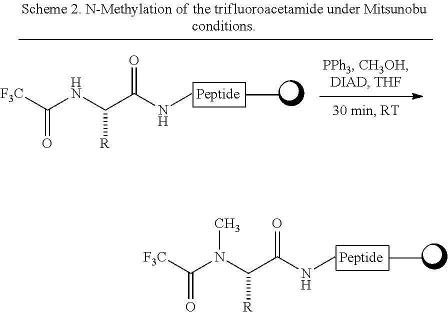

Despite its labile nature as a protecting group, in the context of SPPS, the Tfac protecting group is quite robust. The inventors investigated all of the methods previously reported to cleave the Tfac group in solution-phase chemistry. The reagents commonly used to remove other amino protecting groups, such as piperidine, hydrazine, and 2 mercaptoethanol were found to have no effect on the Tfac group during a 60 min reaction. The only method that successfully removed the Tfac group within 60 min was treatment with excess sodium borohydride in a mixed solvent system of THF and ethanol (1:1 v/v) (Scheme 3).

The results indicate that the Tfac protecting group is orthogonal to other amino protection groups such as Fmoc, Boc, Dde, and o-NBS. In addition, sodium borohydride is known to be compatible with other commonly used side chain protection groups, with the exception of the methyl and allyl esters. Methyl and allyl esters are reduced to the corresponding alcohols by treatment with sodium borohydride, however the t-butyl ester is stable to these conditions.

The stability of the trifluoroacetamide group, in conjunction with its orthogonality to other protection groups, makes it a valuable tool for chemists seeking a means by which to conduct SPPS with the option of methylating backbone amides in the presence of other potentially reactive side chain moieties such as those in lysine and arginine.

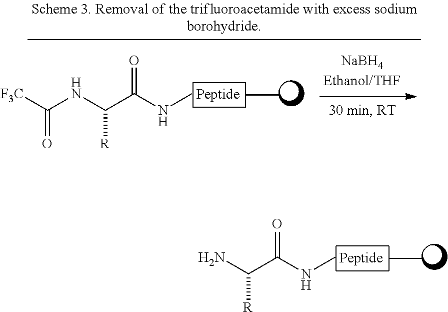

The structure of Tfac (trifluoroacetamide) is as follows:

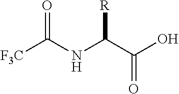

Below is a non-exhaustive list of exemplary amino acids (both L and D forms) shown as their Tfac-protected derivatives.

Tfac-Protected Amino Acids

Tfac-Ala-OH

N-α-Tfac-L-alanine

Tfac-Arg(Pbf)-OH

N-α-Tfac-NG-(2,2,4,6,7-pentamethyldihydrobenzofuran-5-sulfonyl)-L-arginine

Tfac-Asn(Trt)-OH

N-α-Tfac-N-β-trityl-L-asparagine

Tfac-Asp(OtBu)-OH

N-α-Tfac-L-aspartic acid β-t-butyl ester

Tfac-Cys(Trt)-OH

N-α-Tfac-S-trityl-L-cysteine

Tfac-Gln(Trt)-OH

N-α-Tfac-N-γ-trityl-L-glutamine

Tfac-Glu(OtBu)-OH

N-α-Tfac-L-glutamic acid γ-t-butyl ester

Tfac-Gly-OH

N-α-Tfac-glycine

Tfac-His(Trt)-OH

N-α-Tfac-N-im-trityl-L-histidine

Tfac-Ile-OH

N-α-Tfac-L-isoleucine

Tfac-Leu-OH

N-α-Tfac-L-leucine

Tfac-Lys(Boc)-OH

N-α-Tfac-N-ε-t-Boc-L-lysine

Tfac-Met-OH

N-α-Tfac-L-methionine

Tfac-Phe-OH

N-α-Tfac-L-phenylalanine

Tfac-Pro-OH

N-α-Tfac-L-proline

Tfac-Ser(tBu)-OH

N-α-Tfac-O-t-butyl-L-serine

Tfac-Thr(tBu)-OH

N-α-Tfac-O-t-butyl-L-threonine

Tfac-Trp(Boc)-OH

N-α-Tfac-N-in-t-Boc-L-tryptophan

Tfac-Tyr(tBu)-OH

N-α-Tfac-O-t-butyl-L-tyrosine

Tfac-Val-OH

N-α-Tfac-L-valine

Tfac-Protected D-Amino Acids

Tfac-Ala-OH

N-α-Tfac-D-alanine

Tfac-D-Arg(Pbf)-OH

N-α-Tfac-NG-(2,2,4,6,7-pentamethyldihydrobenzofuran-5-sulfonyl)-D-arginine

Tfac-D-Asn(Trt)-OH

N-α-Tfac-N-p-trityl-D-asparagine

Tfac-D-Asp(OtBu)-OH

N-α-Tfac-D-aspartic acid β-t-butyl ester

Tfac-D-Cys(Trt)-OH

N-α-Tfac-S-trityl-D-cysteine

Tfac-D-Gln(Trt)-OH

N-α-Tfac-N-γ-trityl-D-glutamine

Tfac-D-Glu(OtBu)-OH

N-α-Tfac-D-glutamic acid γ-t-butyl ester

Tfac-D-Gly-OH

N-α-Tfac-D-glycine

Tfac-D-His(Trt)-OH

N-α-Tfac-N-im-trityl-D-histidine

Tfac-D-Ile-OH

N-α-Tfac-D-isoleucine

Tfac-D-Leu-OH

N-α-Tfac-D-L-leucine

Tfac-D-Lys(Boc)-OH

N-α-Tfac-D-N-E-t-Boc-L-lysine

Tfac-D-Met-OH

N-α-Tfac-D-L-methionine

Tfac-D-Phe-OH

N-α-Tfac-D-L-phenylalanine

Tfac-D-Pro-OH

N-α-Tfac-D-L-proline

Tfac-D-Ser(tBu)-OH

N-α-Tfac-D-O-t-butyl-L-serine

Tfac-D-Thr(tBu)-OH

N-α-Tfac-D-O-t-butyl-L-threonine

Tfac-Trp(Boc)-OH

N-α-Tfac-N-in-t-Boc-L-tryptophan

Tfac-Tyr(tBu)-OH

N-α-Tfac-O-t-butyl-L-tyrosine

Tfac-Val-OH

N-α-Tfac-L-valine

Tfac-Protected D-Amino Acids

Tfac-Ala-OH

N-α-Tfac-D-alanine

Tfac-D-Arg(Pbf)-OH

N-α-Tfac-NG-(2,2,4,6,7-pentamethyldihydrobenzofuran-5-sulfonyl)-D-arginine

Tfac-D-Asn(Trt)-OH

N-α-Tfac-N-β-trityl-D-asparagine

Tfac-D-Asp(OtBu)-OH

N-α-Tfac-D-aspartic acid β-t-butyl ester

Tfac-D-Cys(Trt)-OH

N-α-Tfac-S-trityl-D-cysteine

Tfac-D-Gln(Trt)-OH

N-α-Tfac-N-y-trityl-D-glutamine

Tfac-D-Glu(OtBu)-OH

N-α-Tfac-D-glutamic acid γ-t-butyl ester

Tfac-D-Gly-OH

N-α-Tfac-D-glycine

Tfac-D-His(Trt)-OH

N-α-Tfac-N-im-trityl-D-histidine

Tfac-D-Ile-OH

N-α-Tfac-D-isoleucine

Tfac-D-Leu-OH

N-α-Tfac-D-L-leucine

Tfac-D-Lys(Boc)-OH

N-α-Tfac-D-N-ε-t-Boc-L-lysine

Tfac-D-Met-OH

N-α-Tfac-D-L-methionine

Tfac-D-Phe-OH

N-α-Tfac-D-L-phenylalanine

Tfac-D-Pro-OH

N-α-Tfac-D-L-proline

Tfac-D-Ser(tBu)-OH

N-α-Tfac-D-O-t-butyl-L-serine

Tfac-D-Thr(tBu)-OH

N-α-Tfac-D-O-t-butyl-L-threonine

1. A method for solid phase peptide synthesis (SPPS) comprising N-terminal protection of an amino acid, the method comprising binding of trifluoroacetamide (Tfac) to the N-terminal of the amino acid.

2. The method of claim 1 further comprising deprotection of the amino acid by the removal of the Tfac group, by treatment with excess sodium borohydride in a mixed solvent system.

3. The novel use of trifluoroacetamide (Tfac) for N-terminal protection of amino acids, dipeptides, peptides, and ploypeptides during solid phase peptide synthesis.

4. A Method for the site specific N-terminal methylation of a resin-bound peptide containing an N-terminal trifluoroacetamide, the method comprising treatment of the peptide with an excess of methanol and triphenylphosphine in tetrahydrofuran, followed by the addition of excess diisopropyl azadicarboxylate therby yielding >99% N-methylation product within 30 minutes.

5. The method of claim 4, wherein the method employs Mitsunobu methylation of resin-bound Tfac-protected peptides.

6. The method of claim 5, the method comprising the steps of: (i) drying Methanol and DIAD prior to use, (ii) rinsing the resin with anhydrous THF, (iii) dissolving Triphenylphosphine in anhydrous THF, (iv) adding anhydrous methanol and (v) transferring to a synthesis vial containing the resin-bound Tfa-protected peptide, and (vi) adding DIAD dropwise with agitation, then after the DIAD addition is complete, (vii) shaking for about 30 min reaction time.

7. The method of claim 6 further comprising repeating the steps (ii) to (vii) at least twice.

8. The method of claim 2 wherein the reaction is complete in 60 min and affords >99% yields of pure trifluoroacetamide product.

9. The method of claim 2 wherein said excess sodium borohydride is provided in a mixed solvent system of THF and ethanol.

10. The method of claim 9 wherein said mixed solvent system comprises THF and ethanol.

11. The method of claim 2 further comprising selectively methylating peptides containing an N-terminal trifluoroacetamide by treatment with excess methanol and triphenylphosphine in tetrahydrofuran, followed by the addition of excess diisopropyl azadicarboxylate.

12. A composition comprising one or more amino acids bound to trifluoracetamide comprising at least the following structure: [chemical structure]
